(19) United States
(12) Patent Application Publication
(10) Pub. No.: US 2012/0172940 A1
(43) Pub. Date: Jul. 5, 2012

(54) Therapeutic Garment

(76) Inventors: Terry L. Wahls, Iowa City, IA (US); Richard Shields, Iowa City, IA (US); Ryan David Feld, Buffalo Grove, IL (US)

(21) Appl. No.: 13/308,327

(22) Filed: Nov. 30, 2011

Related U.S. Application Data
(60) Provisional application No. 61/418,127, filed on Nov. 30, 2010.

(51) Int. Cl. A61N 1/375; A41D 1/00

(52) U.S. Cl. 607/3

(57) ABSTRACT

Several embodiments are provided for a modular therapeutic garment having electrodes integral with the garment and treatment permitted by such garment. The therapeutic garment can comprise a compression knit fabric material, and an array of electrodes integral with the compression knit fabric material. The compression knit fabric material can exert pressures within a range including values suitable for clinical treatment. The array of electrodes can be arranged to overlie specific portions of a subject, such as muscle groups or other body parts. Each electrode of the array of electrode can be formed of a conductive knit fabric material. The conductive knit fabric material can enclose a liquid absorptive material. A method for treating a musculoskeletal dysfunction with the therapeutic garment also is provided. The method also can be effective at improving musculoskeletal condition of healthy subjects.

### CROSS-REFERENCE TO RELATED APPLICATION

This patent application relates to and claims priority from U.S. Provisional Application No. 61/418,127, filed on Nov. 30, 2010, which is incorporated by reference herein in its entirety.

### BACKGROUND

Therapeutic compression has been shown to reduce risk of muscle injury, improve recovery time, and improve eccentric motor control. In addition, neuromuscular Electrical Stimulation (NMES) has been used in the past in the treatment of neurodegenerative disorders such as stroke, cerebral palsy, multiple sclerosis and Parkinson's disease and the rehabilitation of athletes. NMES is used to electrically stimulate muscle contractions to strengthen the muscles of patients that are too debilitated to partake in traditional exercise. Electrical stimulation of muscle recruits motor units in reverse order from voluntary muscle contraction. High voltage therapy can be utilized to treat edema, and transcutaneous electrical therapy can be utilized to treat chronic pain.

Neuromuscular electrical stimulation (NMES)-augmented physical training using 80 Hz alternating current for about 15 minutes for strength, and 25 Hz alternative current for about 60 minutes for endurance training, have resulted in mean 5 centimeter increase in vertical jump after 5 weeks of training NMES has also demonstrated improved strength and reduced spasticity in children with cerebral palsy, stroke patients, patients with advanced heart failure, advanced chronic lung disease, rheumatoid arthritis, and multiple sclerosis patients. In addition, NMES has improved endothelial function in patients with refractory heart failure. Age related loss of muscle mass and strength generally leads to loss of function and increasing frailty in the elderly. Yet, through NMES-augmented exercise programs, such loss of function and related frailty may be reversed. NMES-augmented exercise programs can enhance cardiovascular capacity in healthy physically active adults. Even though NMES has been used effectively to allow patients to regain muscle function, the treatment can be inconvenient for a patient to use.

### SUMMARY

Some embodiments of the disclosure relate to a modular therapeutic garment having electrodes integral with the garment and treatment permitted by such garment. The therapeutic garment can combine two technologies for therapy: compression and electrical stimulation. By combining such proven technologies, the therapeutic garment can permit intervention that can benefit healthy subjects and subjects having medical conditions affecting their musculoskeletal system. In one embodiment, the therapeutic garment can comprise a compression knit fabric material, and an array of electrodes integral with the compression knit fabric material. The compression knit fabric material can exert pressures within a range that includes values suitable for clinical treatment (e.g., pressure ranging from at least 50 mmHg). The array of electrodes can be arranged to overlie specific portions of a subject, and each electrode of the array of electrode being formed of a conductive knit fabric material.

According to another aspect, an exemplary method for electrotherapy is provided. The method can be applied to treating a musculoskeletal dysfunction or to improving musculoskeletal condition of healthy subjects. Such method can comprise providing an array of electrodes integral with a compression knit fabric material, wherein the array of electrodes can be arranged to overlie a muscle group of a subject, and each electrode of the array of electrodes being formed of a conductive knit fabric material and a liquid absorptive material. In another aspect, the exemplary method can comprise applying electric stimulation to each electrode of the array of electrodes according to a prescribed treatment. Application of the electric stimulation can comprise applying a voltage waveform, a current waveform, or a combination of both.

Additional aspects, features, or advantages of the subject disclosure will be set forth in part in the description which follows, and in part will be obvious from the description, or may be learned by practice of the subject disclosure. The advantages of the subject disclosure will be realized and attained by means of the elements and combinations particularly pointed out in the appended claims. It is to be understood that both the foregoing general description and the following detailed description are exemplary and explanatory only and are not restrictive of the subject disclosure.

### BRIEF DESCRIPTION OF THE DRAWINGS

The accompanying drawings are incorporated and illustrate exemplary embodiment(s) of the subject disclosure and together with the description and claims appended hereto serve to explain various principles, features, or aspects of the subject disclosure.

FIG. 1 illustrates an exemplary therapy environment in accordance with one or more aspects of the disclosure.

FIG. 2 illustrates an exemplary embodiment of a therapeutic garment in accordance with one or more aspects of the disclosure.

FIGS. 3A-3C illustrate front, rear, and side views of an exemplary electrode configuration for a therapeutic garment in accordance with aspects of the subject disclosure.

FIG. 4 illustrates an exemplary embodiment of a therapeutic garment in accordance with one or more aspects of the disclosure.

FIGS. 5-6 illustrate an exemplary embodiment of a lead wire that can be part of a therapeutic garment in accordance with one or more aspects of the disclosure.

FIG. 7 illustrates exemplary embodiments of therapeutic garments in accordance with one or more aspects of the disclosure.

FIG. 8 is a flowchart of an exemplary method in accordance with one or more aspects of the subject disclosure.

### DETAILED DESCRIPTION

The present disclosure may be understood more readily by reference to the following detailed description of exemplary embodiments of the subject disclosure and to the figures and their previous and following description.

Before the present garments, articles, devices, and/or methods are disclosed and described, it is to be understood that the subject disclosure is not limited to specific systems and methods for a modular therapeutic garment having electrodes integral with the garment and treatment therewith. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats and that this data represents endpoints and starting points, and ranges for any combination of the data points. For example, if a particular data point “10” and a particular data point “15” are disclosed, it is understood that greater than, greater than or equal to, less than, less than or equal to, and equal to “10” and “15” are considered disclosed as well as between “10” and “15.” It is also understood that each unit between two particular units are also disclosed. For example, if “40” and “50” are disclosed, then “41,” “42,” “43,” “44,” “45,” “46,” “47,” “48,” and “49” also are disclosed.

In the subject specification and in the claims which follow, reference may be made to a number of terms which shall be defined to have the following meanings: “Optional” or “optionally” means that the subsequently described event or circumstance may or may not occur, and that the description includes instances where said event or circumstance occurs and instances where it does not.

As employed in this specification and annexed drawings, the terms “unit,” “component,” “interface,” “system,” and the like are intended to include a computer-related entity or an entity related to an operational apparatus with one or more specific functionalities, wherein the computer-related entity or the entity related to the operational apparatus can be either hardware, a combination of hardware and software, software, or software in execution. One or more of such entities also are referred to as “functional elements.” As an example, a unit may be, but is not limited to being, a process running on a processor, a processor, an object, an executable computer program, a thread of execution, a program, a memory (e.g., a hard disc drive), and/or a computer. As another example, a unit can be an apparatus with specific functionality provided by mechanical parts operated by electric or electronic circuitry which is operated by a software or a firmware application executed by a processor, wherein the processor can be internal or external to the apparatus and executes at least a part of the software or firmware application. In addition or in the alternative, a unit can provide specific functionality based on physical structure or specific arrangement of hardware elements. As yet another example, a unit can be an apparatus that provides specific functionality through electronic functional elements without mechanical parts, the electronic functional elements can include a processor therein to execute software or firmware that provides at least in part the functionality of the electronic functional elements. The foregoing example and related illustrations are but a few examples and are not intended to be limiting. Moreover, while such illustrations are presented for a unit, the foregoing examples also apply to a component, a system, a platform, and the like. It is noted that in certain embodiments, or in connection with certain aspects or features thereof, the terms “unit,” “component,” “system,” “interface,” can be utilized interchangeably.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

In the present specification and annexed drawings, the term “subject” refers to living organisms, either human or a non-human animal, of various physiologies—e.g., specific nervous tissue organization; specific musculoskeletal system, such as muscle groups and supporting bone(s) and connective tissue(s); and so forth. Examples of a non-human animal include but are not limited to a pig, monkey, chimpanzee, orangutan, cat, dog, sheep, or cow. A subject can be a natural animal or a transgenic non-human animal including but not limited to a transgenic mouse or transgenic rat.

Reference will now be made in detail to the various embodiment(s), aspects, and features of the subject disclosure, example(s) of which are illustrated in the accompanying drawings. Wherever possible, the same reference numbers are used throughout the drawings to refer to the same or like parts.

The disclosure identifies and addresses issues with implementation of NMES therapy, TENS therapy, high-voltage therapy for edema, microcurrent therapy for treatment of cellular function, mitochondrial function and improved tissue repair. Such therapy generally requires the patient to use electrodes throughout the day which involves switching the placement of electrodes frequently to stimulate different muscle groups. This also means that the patient must place the electrodes on his or her own, making it difficult to stimulate the correct muscle groups. Conventional electrodes can also be brittle and uncomfortable to wear. Free lead wires can easily become tangled, leading to pull away of electrodes or detachment of the lead wire from the electrodes. To ensure proper skin contact, the electrodes must be bound on with constraining compression straps.

As described in greater detail below, a therapeutic garment of the disclosure can combine two proven technologies for therapy: compression and electrical stimulation. By combining such proven technologies, the therapeutic garment of the disclosure can permit intervention that can benefit healthy subjects (e.g., adults or children) wishing to improve their muscle strength or endurance. It should be appreciate that by combining volitional muscle contraction with electrical stimulation of the same muscle, shortened rehabilitation time is achieved. In addition or in the alternative, the therapeutic garment can permit intervention of subjects having muscle weakness due to clinical conditions (e.g., chronic disease or physiologic age related muscle loss) and wishing to improve of physical performance. Furthermore, or as another alternative, the disclosed therapeutic garment of the disclosure can be employed clinically, e.g., as part of a therapy, for treatment of various medical conditions. For example, the therapeutic garment can be utilized to augment voluntary exercise programs to aid in recovery following injury or to improve muscle strength and endurance in response to the setting of a chronic disease or age related sarcopenia. The therapeutic garment can be utilized as an instrument of preventive medicine.

Some embodiments of the disclosure provide a system of electrodes manufactured of conductive and within modular compression components, which can be tailored to specific subject's neuromuscular electrical stimulation needs (e.g., which muscle groups may require stimulation). The therapeutic garment can interface with commercially available electrical therapy units (ETUs), either portable or stationary, to permit electrotherapy for treatment of generalized deconditioning from medical conditions, such as neuromuscular disease; improved muscle endurance and muscle strength; increased bone density; improved nerve growth factors; improved endorphin levels; decreased atrophy of muscle and decreased loss of bone mineral during periods of exposure to low gravity environments or weightlessness; diminished risk of blood stagnation; treatment of muscle atrophy and diminished bone mineralization during prolonged immobility, such as long travel. The electrotherapy can comprise a progressive strength training program.

In certain embodiments, the therapeutic electrode garment can permit treatment (via compression and electrical therapy, for example) in subjects affected by neurodegenerative disorders, edema, and the like. In other embodiments, the therapeutic electrode garment can permit electrical therapy for electrical stimulation of muscle contractions in subjects affected by low gravity environments, prolonged immobility, or otherwise deconditioning environments, and in healthy subjects training muscles for enhanced strength and endurance.

The therapeutic garment of the disclosure can permit electric stimulation of various parts (e.g., muscle groups) of a subject without excessive adjustment or reconfiguration of electrodes throughout a therapy session, or treatment period. For instance, electrodes of the therapeutic garment can be adjusted or configured at the beginning of a therapy session, without subsequent adjustment or reconfiguration. In one embodiment, the therapeutic garment can operate in a configuration that eliminates free lead wires dangling in space and the ensuing risk of tangling with each other and other objects, resulting in pulling the electrodes away from the skin and causing sharp jumps in pain as that happens.

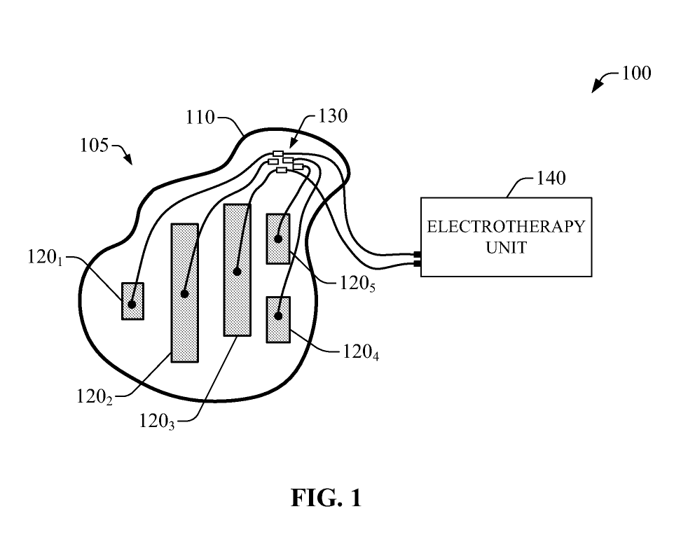

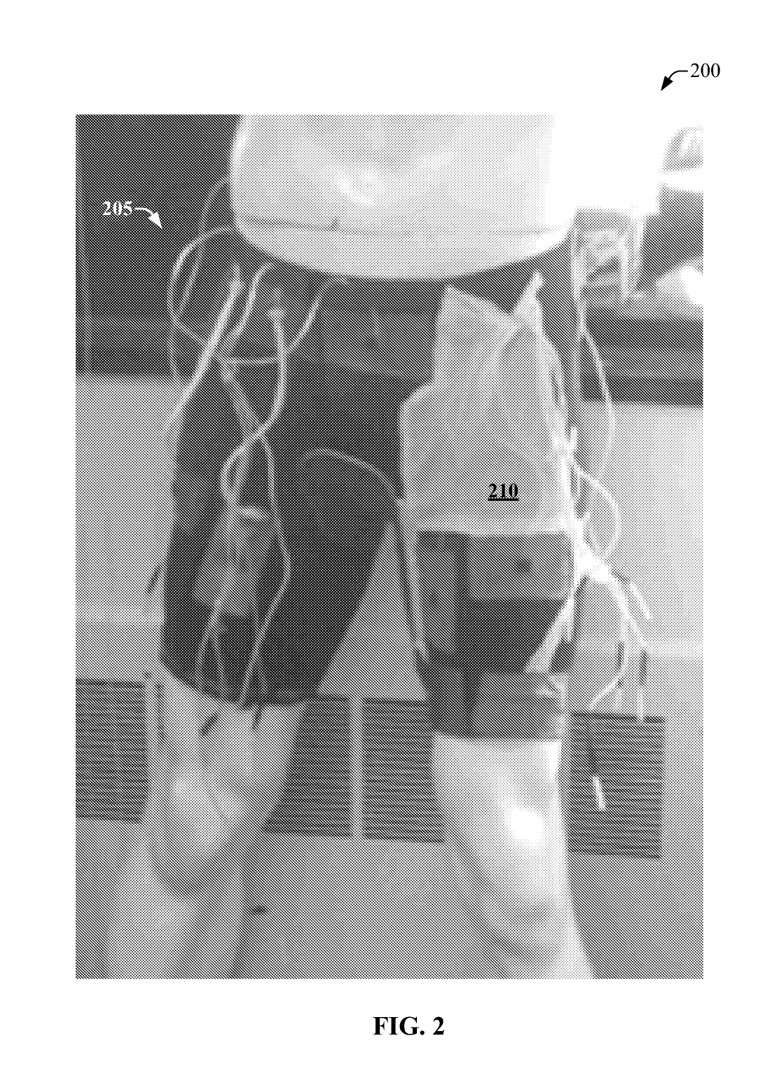

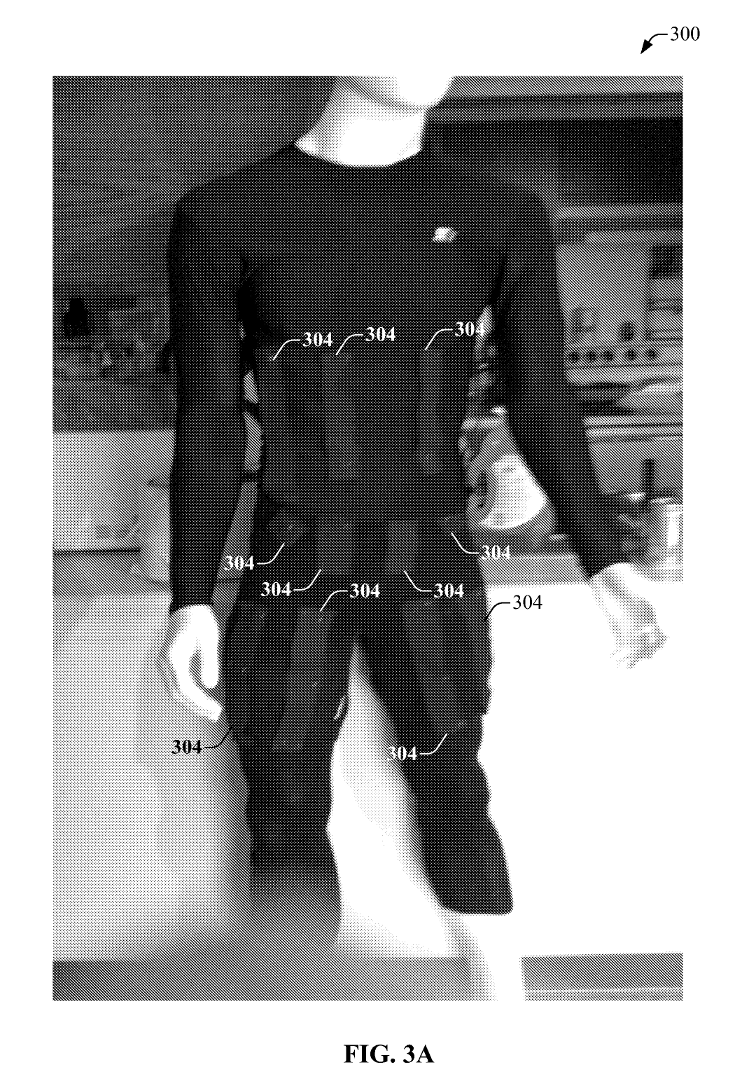

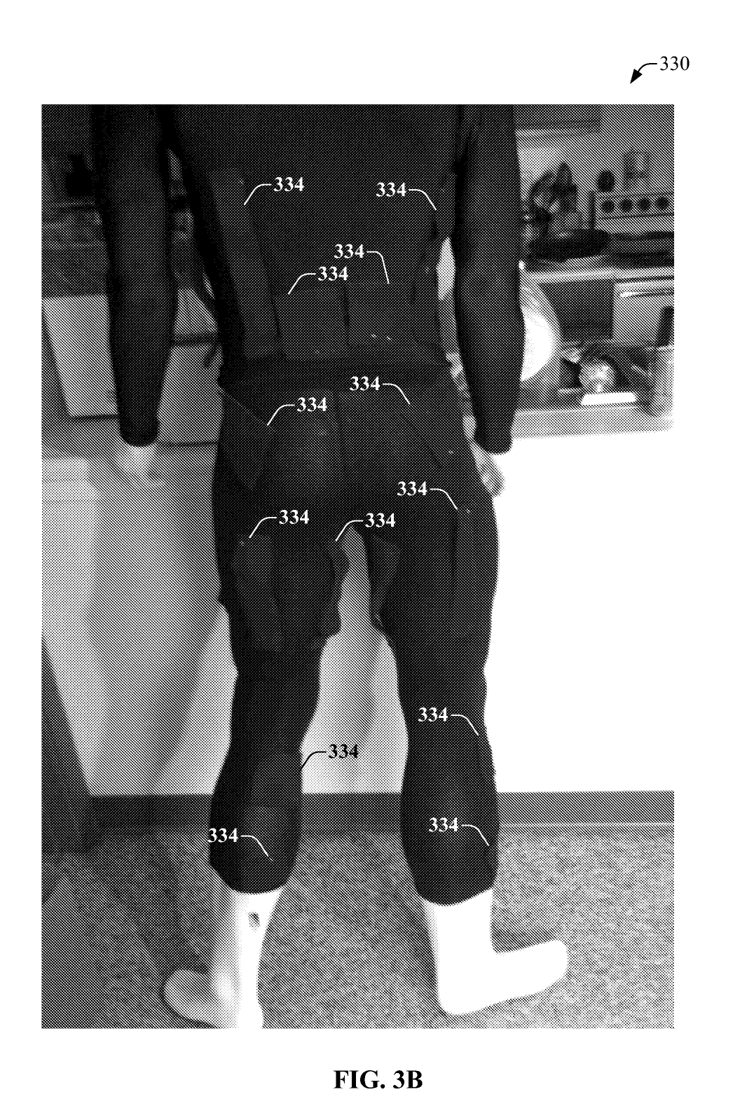

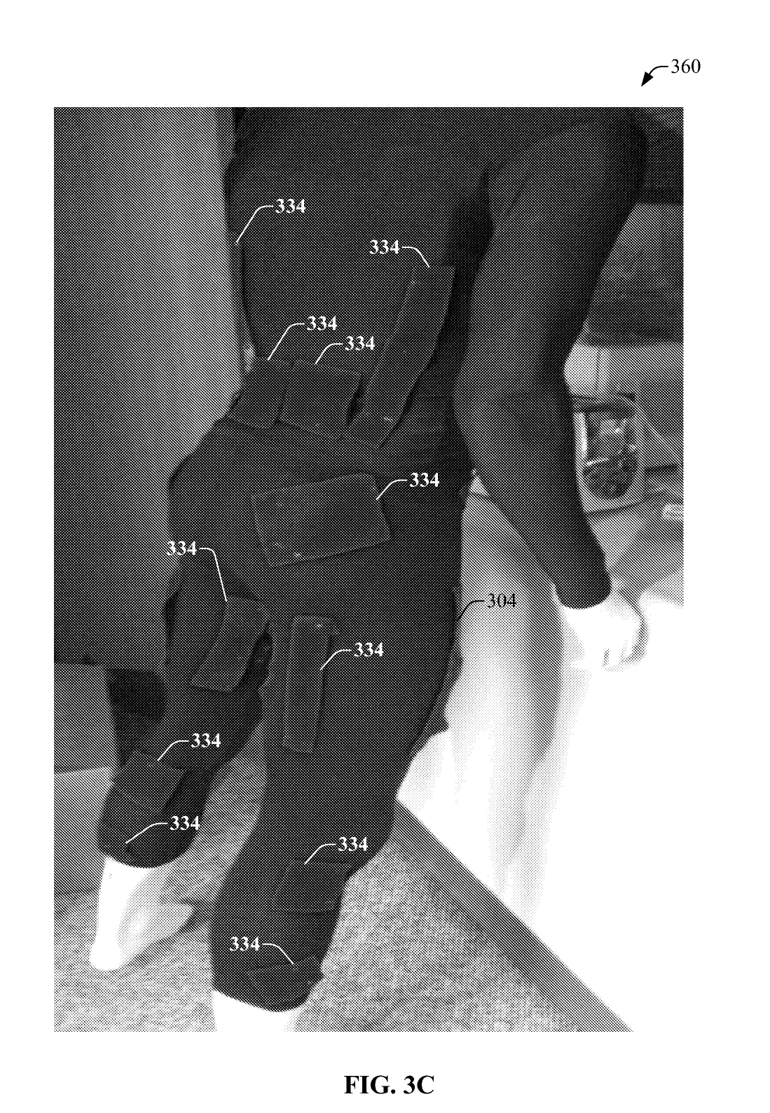

FIG. 1 illustrates an exemplary therapy environment 100 in accordance with one or more aspects of the disclosure. The therapy environment 100 includes a therapeutic garment 105 comprising a compression knit fabric material 110, and an array of electrodes 120₁-120₅ integral with the compression knit fabric material 110. While the illustrated array of electrodes (represented with grey rectangles in FIG. 1) includes five electrodes, the disclosure is not so limited and other number of electrodes can compose the array of electrodes (see, e.g., FIG. 2 or FIG. 3A-3C (in which electrodes 304 and electrodes 334 are shown as dark grey portions on top of an underlying compressive knit fabric shown in black). As illustrated in FIGS. 3A-3C, the array of electrodes can be arranged to overlie specific portions of a subject, and each electrode of the array of electrode being formed of a conductive knit fabric material. The electrode arrangement can permit, at least in part, placement of the therapeutic garment 105 on a subject, allowing adjustment of the therapeutic garment in a manner that the electrodes lie comfortably onto the body part to be stimulated. For a human subject, in one aspect, such portions can comprise a plurality of areas in a muscle group. In another aspect, the specific portions of the subject can comprise arm(s), leg(s), torso, neck, or the like. In one aspect, an electrode of the array of electrodes can be sized according, at least in part, to physiology of the specific portion of the subject, e.g., structure of a muscle group to be treated (e.g., electrically stimulated). In one embodiment, each of the electrodes in the array of electrodes 120₁-120₅ can be sewn into the compression knit fabric material 110 to overlie the motor points (or motor neurons) of a specific muscle group. In another embodiment, one or more of the electrodes of the array of electrodes 1201-1205 can sprayed, or otherwise, coated onto the compression knit fabric material 110.

Each electrode in the array of electrodes is electrically conductive (e.g., it can transport an electrical current). The electrically conductive knit material of each electrode of the array of electrodes can be a stretchable knit fabric material. For example, such materials can comprise materials having one or more of a combination of cotton fibers and electrically conductive threads (e.g., metal threads such as silver threads, gold threads, aluminum threads, etc.), a combination of synthetic fibers and metal threads, a combination of bamboo fibers and metal threads, a combination of linen fibers and metal threads, a combination of wool fibers and metal threads, or any variations of such combinations. In one embodiment, the therapeutic garment 105 can utilize commercially available conductive cloth for one or more electrodes in the array of electrodes 120₁-120₅. Selection of a commercially available conductive cloth can be based at least in part on one or more of electrical conductivity of such cloth or comfort. In another embodiment, the electrically conductive knit material can be a bamboo based fabric having silver fibers woven in. Such fabric can be the bamboo electrically conductive cloth, containing silver fibers, that is manufactured by Less EMF Inc. of Albany, N.Y. A bamboo based fabric having electrically conducting fibers can be comfortable to a subject undergoing treatment since such a fabric lacks scratchy fibers that can irritate the subject's skin while the conducting fibers can conduct sufficient electricity to stimulate, for example, a muscle contraction. A heavier compression fabric can be utilized for the non-electrode portions of therapeutic garment as such fabric can be more durable and can exert higher compression than a thinner compression fabric. It should be appreciated that thin compression fabrics can lose elasticity more rapidly than heavier compression fabrics. Other suitable materials for the electrodes and other portions of the disclosed therapeutic garments can be utilized.

In one aspect, each of the electrodes in the array of electrodes 120₁-120₅ can be moistened to maintain good electrical contact between the electrode and skin and thus provide low resistance and comfort for a treated subject. Accordingly, in certain embodiments, the conductive knit fabric material of each electrode of the array of electrodes can encompass at least a portion of a liquid absorptive material, such as a sponge.

At least one dimension of each electrode in the array of electrodes 120₁-120₅ can be determined at least in part by the specific portion of the subject (e.g., a muscle group of the subject) to be treated. Accordingly, each electrode of the array of electrodes can have specific dimensions. In one embodiment of the therapeutic garment 105, e.g., embodiment 200 in FIG. 2, at least two electrodes can have substantially the same size. Symmetry considerations of a muscle group can determine the number of electrodes in an array of electrodes that can have substantially the same size. In exemplary embodiment 200, the therapeutic garment 205 is shown fitted on a mannequin. The therapeutic garment 205 is a shorts module, and is manufactured from a compression knit fabric material having synthetic fibers in a tight knit. In one aspect, the compression minimizes muscle oscillations and improves blood flow return. In the therapeutic garment 205, the conductive knit fabric material for the electrodes (represented with grey blocks in FIG. 2) is a knit fabric having spandex, for elasticity (e.g., compression or expansion), and silver fibers for conductivity. A rivet (shown as a black dot in FIG. 1 and FIG. 2) attached to each electrode permits connection of an insulated electrical wire to the electrode. The wire then has an interfacing sleeve to connect with the pins making the shorts module a universally compatible therapeutic electrode garment. In one aspect, presence of such sleeve can permit the therapeutic garment 205 to interface with any ETU. The electrodes in therapeutic garment 205 can be placed over motor points to stimulate the targeted muscle groups.

The compression knit fabric 110 utilized for the construction of the therapeutic garment 105 can have one or more of the following properties: moderate compression (e.g., exerted pressures from about 10 mmHg to about 15 mmHg); clinical compression (e.g., exerted pressures from about 15 mmHg to about 65 mmHg); breathability (e.g., the fabric permits transmission of water vapor); hypoallergenic; non-abrasive; washable in warm water; antibacterial; antiodor; or lightweight while maintaining compression. It should be appreciated that compression can exhibit a gradient among two or more compression values within the disclosed compression ranges. Such gradient can represent a spatial variation of compression. Other suitable characteristics of the compression knit fabric also can be exploited based at least in part on therapy needs of the user. In one aspect, the therapeutic garments of the disclosure can be manufactured with high-end compression materials, such as materials that exert pressure (e.g., compression) levels of at least 10 mm Hg. In another aspect, the therapeutric garments of the disclosure can be manufactured with a clinical-grade compression knit fabric material, which can exert pressures ranging from about 30 mmHG to about 40 mmHg. Such compression in combination with NEMS can be suitable for treatment of lymphedema, improving removal of fluid from a subject's extremities, for example. Similar clinical-grade compression knit fabric materials providing pressures of at least 50 mmHg (e.g., 50 mmHg, 51 mmHg, 52 mmHg, 53 mmHg, 54 mmHg, 55 mmHg, 56 mmHg, 57 mmHg, 58 mmHg, 59 mmHg, 60 mmHg, 61 mmHg, 62 mmHg, 63 mmHg, mmHg 64 mmHg, 65 mmHg) also can be employed in manufacture of the therapeutic garments of the disclosure. For certain knit fabrics, the level of compression that can be exerted can be regulated by adjusting amounts of materials that compose the knit fabrics. For example, for Spandex™, the amount of nylon fibers and the amount of polyester can determine the attainable pressure levels. In one embodiment, the compression knit fabric material 110 can comprise one or more of a material configured (e.g., manufactured) to transmit water vapor, an antibacterial material, a hypoallergenic material, or a non-abrasive material.

The compression knit fabric material 110 of therapeutic garment 105 can be manufactured exert pressure in the range from about 10 mm Hg to about 60 mm Hg depending, at least in part, on the location within the therapeutic garment. In one embodiment, the therapeutic garment can be a body suit (see, e.g., FIGS. 3A-3B) that can be worn by a human being. Such therapeutic garment can cover legs, torso, and arms. In another embodiment, the therapeutic garment 105 can be modular, comprising one or more units, such as an arm sleeve or a leg sleeve.

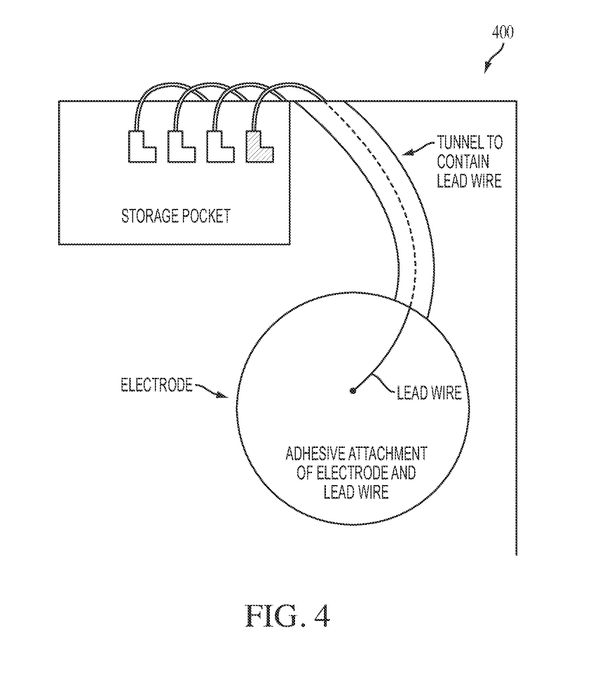

The therapeutic garment 105 can comprise a rivet (represented with a black circle in FIG. 1) for each electrode of the array of electrodes. The rivet can be sewn or riveted into such garment. The rivet can be configured (e.g., machined or molded) to attach a conducting (represented with a solid curved line) wire to an electrode associated with the rivet. The conducting wire can be referred to as a lead wire. In one embodiment, a lead wire can be attached to at least one electrode of the array of electrodes by using an adhesive (e.g., a nominally permanent epoxy adhesive) which permits conduction of electrical current into the electrode. In such embodiment, a lead wired is referred to as being adhesively attached to a respective electrode. Other means for coupling, such as sewing, can be utilized to attach securely a lead wire to an electrode of the array of electrodes. In one embodiment, a lead wire can be sewn into the therapeutic garment 105. In another embodiment, e.g., embodiment 400 in FIG. 4, a lead wire can traverse the therapeutic garment 105 enclosed in a tunnel of the same compression knit fabric material 110 employed to manufacture such garment.

As described herein (see, e.g., FIG. 2), a therapeutic garment of the disclosure (e.g., therapeutic garment 105 or therapeutic garment 205) can comprise a sleeve (e.g., element 210 in FIG. 2) housing at least one (e.g., one, two, more than two, . . . ) conductive wire attached to at least one (e.g., one, two, more than two, . . . ) electrode of the array of electrodes. The sleeve can be attached to an electrode that can interface universally with an ETU. In one embodiment, the sleeve can be a mesh sleeve (e.g., element 210 in FIG. 2) that can contain one or more lead wires along the lateral seams of the therapeutic garment of the disclosure (e.g., therapeutic garment 105 or therapeutic garment 205) for best comfort and best control of loose wires.

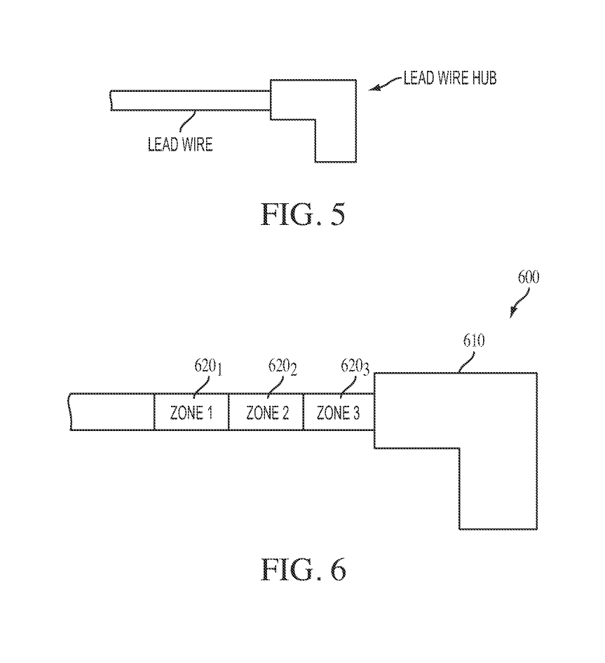

As illustrated in FIG. 1 and in FIG. 5, a lead wire attached to an electrode (e.g., electrode 120₂) of a therapeutic garment can have a hub at a distal end from the electrode. In the therapy environment 100, five hubs are illustrated, forming a group of hubs 130. It should be appreciated that the five hubs are illustrative and the disclosure is not so limited. As illustrated in FIG. 2, in certain embodiments, more than five hubs can be present in a therapeutic garment. Each hub (represented with a rectangle in FIG. 1) in the group of hubs can interface the therapeutic garment 105 with an electrotherapy unit 140 (also referred to as electric therapy unit 140). Such unit can be either portable or stationary and, in certain implementations, it can be a commercially available electrical therapy unit, such as the Empi® 300PV™ manufactured by Empi® of St. Paul, Minn., which has FDA approved indications for the delivery of both neuromuscular electrical stimulation (for muscle atrophy, muscle spasm, acute muscle pain, subacute muscle pain, chronic muscle pain) and transcutaneous electrical stimulation (TENS) for pain management, and high voltage for the treatment of edema. In certain embodiments, the therapeutic garment 105 can have one or more couplings, rather than hubs, that permit interfacing to a commercially available ETU (or electrical stimulator). Such couplings can include, for example, RF connectors, serial connectors, rivets which can serve as snap-to-pin converters, or the like.

When not in use, a hub of the group of hubs 130 can be stored in a storage portion of the therapeutic garment. For instance, as illustrated in FIG. 4, the storage portion can be a pocket at the waist of a short module or a pocket placed at suitable areas of other modules having space to include pocket(s), such as a shirt module (for torso), a pants module (for short leg or long leg), glove module (for a hand), socks module (for a foot), facial module, or an arm module having a pocket at an upper arm area or at the wrist area.

In one embodiment, e.g., exemplary embodiment 600 in FIG. 6, a hub (e.g., hub 610) in the group of hubs 130 can be color-coded to convey association which a muscle group or, more generally, with a body part (e.g., arm, leg, or torso) that can be treated or stimulated in response to connection of a hub to an ETU. For example, for a human subject, Color Zone 2 620₂ can indicate arm(s), leg(s), and torso, whereas Color Zone 3 620₃ can indicate a specific muscle group (e.g., calves) or specific body part. Color coding also can be utilized to indicate a location within the subject's body, such as Right or Left, as indicated by Color Zone 3 620₁. It should be appreciated that more or less zones than three also are contemplated. Such color coding can permit convenient access and identification of muscle groups associated with each hub. Selected hubs (or other couplings) can be connected to the electrotherapy unit 140, which can comprise a plurality of channels and associated connectors for delivery, or application, of current waveforms, voltage waveforms, or both. In the exemplary environment 100, as an illustration, the electrotherapy unit 140 has two channels available. Accordingly, two body parts associated with two electrodes, e.g., 120₁ and 120₃, can be electrically stimulated with the electrotherapy unit 140. Other labeling or encoding (numerals, letters, alphanumeric labels, tokens, etc.) also can be employed.

In certain embodiments, lead wires attached to the therapeutic garment 105 may not be able to be practically replaced in case the wires fail or break. Accordingly, selection of wire materials can be based on durability, for example, with selected materials being expected to last for at least the duration of the therapeutic garment under operation conditions. In one aspect, lead wires utilized in conventional clothing products (e.g., women undergarment) can be inadequate in view of the thickness of such wires and durability thereof as compared to expected duration of the therapeutic garment. Accordingly, the disclosed therapeutic garments can utilize lead wires with lower (or thicker) gauges than those of traditional lead wires. As an example, lead wires with gauges ranging from about 18 to about 28 can be utilized. Wires having other suitable gauges also can be employed. In other embodiments, therapeutic garments can be manufactured in a manner that permits replacement one or more lead wires attached to the garments. In such embodiments, thickness and durability of the lead wires can be less critical when selecting suitable wire materials. In one aspect, gauge of the wires can be selected based at least on voltage and current parameters of an electric therapy unit utilized for electric stimulation.

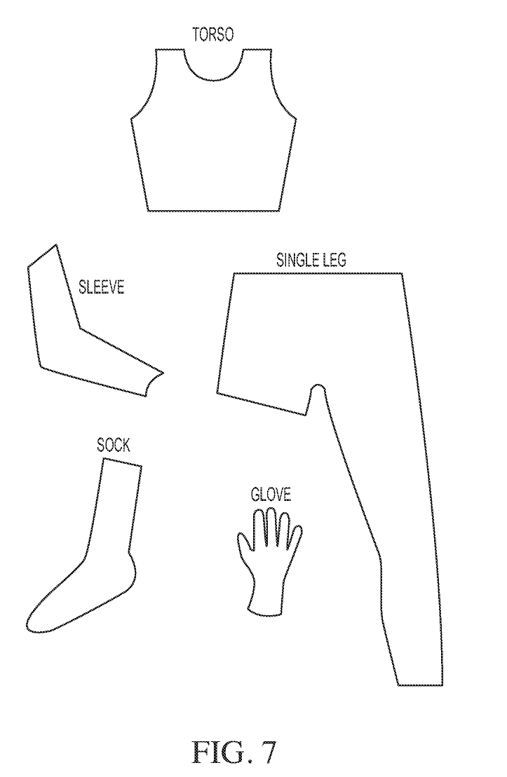

The therapeutic garment 105 can be modular and configured to treat a specific part of the subject. As illustrated in FIG. 7, the therapeutic garment can be modular in that the garment can fit a specific body part of a subject, such as arm, leg, hand, torso, foot, and the like. Accordingly, a therapeutic garment can be a module directed to treatment of a particular body part, such as torso, an arm, a leg, buttocks, thighs, or the like. In addition or in the alternative, the therapeutic garment also can be regarded as modular in that two or more modules (e.g., a shirt module for torso and arms and shorts for both legs) can compose such therapeutic garment. Thus, a therapeutic garment can be manufactured to treat both arms in a human; both legs in a human, or two legs in a race horse; buttocks and thighs in a human, and the like. In one aspect, the modularity of therapeutic garments of the disclosure can permit a therapeutic garment to be tailored (e.g., by combining two or more modules) to permit a subject (a human being, a race horse, etc.) to have the therapeutic garment adapted to a specific treatment or therapy, without incorporating unnecessary modules (or components) into the therapeutic garment, such modules typically providing no therapeutic value and adding unnecessary weight.

Several muscle groups can be treated through compression and electric stimulation via the therapeutic garments of the disclosure. Therapeutic garments can include tights, shorts, arm sleeves (short and long), shirts, tanks, facial masks, helmets, among other garment modules. In one aspect, shorts and tights can permit treatment (e.g., electric stimulation) of muscles comprising one or more of gluteus (maximus and medius), hamstring (biceps femoris), or quads (quadriceps). In another aspect, tights can permit treatment of calves (gastrocnemius). In yet another aspect, a long sleeve module of the disclosed therapeutic garment can permit treatment of muscles comprising one or more of traps (trapezius), triceps (triceps brachii), shoulders (deltoids), lats (latissimus dorsi), middle back (rhomboids), biceps (biceps brachii), lower back, forearm (brachioradailis), or abs (rectus abdominis). In another aspect, a shirt module of the disclosed therapeutic garment can permit treatment of muscles comprising one or more of traps (trapezius), shoulders (deltoids), lats (latissimus dorsi), middle Back (rhomboids), lower back, or abs (rectus abdominis). In still another embodiment, a tank top module of the disclosed therapeutic garment can permit treatment of muscles comprising one or more of traps (trapezius), lats (latissimus dorsi), middle back (rhomboids), lower back, abs (rectus abdominis).

In one aspect, healthy adults and children using the disclosed therapeutic garment with a NMES augmented progressive exercise program can achieve, among other things, (1) more rapid gains in strength and endurance, (2) lower incidence of muscular injury, (3) reduction of risk of chronic constipation, and (4) more rapid recovery following muscular injury. In addition or in the alternative, healthy adults undergoing low gravity environments or immobility (e.g., long flights) can reduce the risk of blood clots in lower extremities and disuse muscle atrophy by using the disclosed therapeutic garment with a NMES augmented progressive exercise program.

In another aspect, healthy elderly adults who wish to prevent or regress age related loss of muscle mass, using the disclosed therapeutic garment with a NMES augmented progressive exercise program can achieve, among other things, (1) gains in strength and endurance, (2) decrease the rate of age related muscle loss, and (3) preservation of functional ambulation (e.g., stair climbing), writing, typing, or more generally, keyboarding, and independent living.

By using the disclosed therapeutic garment and being treated with a NMES augmented progressive exercise program via such garment, adults and children having deconditioned muscles due to a chronic disease, such as severe heart failure, chronic lung disease, rheumatoid arthritis, severe osteoarthritis, diabetes, obesity, other chronic diseases, or any combination of the foregoing, can experience, among other things, (1) improved strength and endurance by using the disclosed therapeutic garment with a progressive exercise program. Likewise, through treatment with a NMES augmented progressive exercise program via the therapeutic garment of the disclosure, adults and children having a chronic disease and suboptimal strength endurance due to neuromuscular disorders such as stroke, cerebral palsy, Parkinson's disease, and multiple sclerosis can, for example, (1) improve muscle strength and endurance, (2) prevent or retard muscle atrophy which would otherwise be expected from their disease process, (3) reduce muscle spasm and stiffness, (4) improve endolethial function, and (5) maintain (in those with disorders anticipated to progress as in multiple sclerosis) and improve function of legs, arms, trunk, etc., with the ensuing preservation of mobility (e.g., standing up from a seated position, utilizing toilets and lavatories, climbing and descending stairs, or the like).

In one aspect, by using the disclosed therapeutic garment with a NMES augmented progressive exercise program, subjects having spinal cord injuries or disuse atrophy, or experiencing low-gravity environments can achieve, among other things, (1) preservation of bone integrity, and (2) reduction of risk of osteoporosis and bone fracture.

In another aspect, by using the disclosed therapeutic garment with a high-voltage treatment enabled by the therapeutic garment of the disclosure, subjects can achieve, among other things, (a) reduced risk of edema, (b) reduced risk of venous pooling, and (c) reduced risk of venocclusive disease.

In yet another aspect, by using the disclosed therapeutic garment with a microcurrent treatment enabled by the therapeutic garment of the disclosure, subjects can achieve, among other things, (a) reduced risk of chronic stasis ulcers, and (b) increased healing rate of wounds or ulcers.

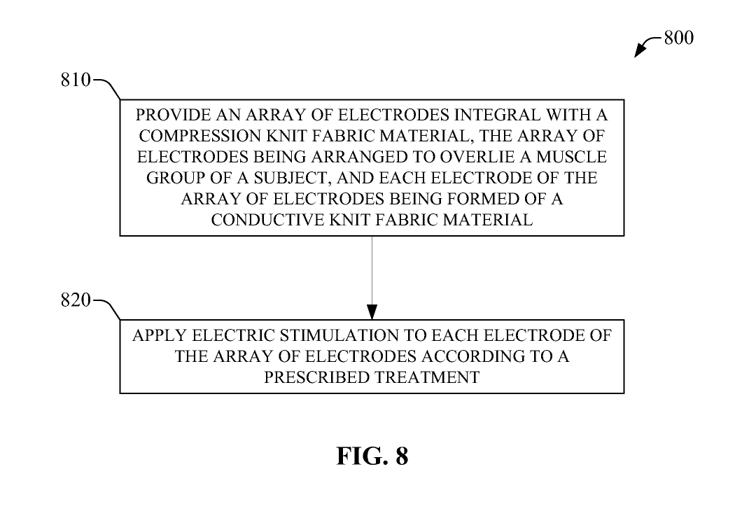

In view of the aspects described hereinbefore, an exemplary method that can be implemented in accordance with the disclosed subject matter can be better appreciated with reference to the flowchart in FIG. 8. For purposes of simplicity of explanation, the exemplary method disclosed herein is presented and described as a series of acts; however, it is to be understood and appreciated that the claimed subject matter is not limited by the order of acts, as some acts may occur in different orders and/or concurrently with other acts from that shown and described herein. For example, the various methods or processes of the disclosure can alternatively be represented as a series of interrelated states or events, such as in a state diagram. Moreover, when disparate functional elements implement disparate portions of the methods or processes in the subject disclosure, an interaction diagram or a call flow can represent such methods or processes. Furthermore, not all illustrated acts may be required to implement a method in accordance with the subject disclosure. Further yet, two or more of the disclosed methods or processes can be implemented in combination with each other, to accomplish one or more features or advantages herein described.

FIG. 8 is a flowchart of an exemplary method 800 for electrotherapy in accordance with aspects of the subject disclosure. In one aspect, the exemplary method 800 can be effective to treat a musculoskeletal medical condition. In another aspect, the exemplary method 800 can be effective at improving musculoskeletal condition of healthy subjects. The therapeutic garment of the disclosure can be employed for implementation of the exemplary method 800. At block 810, an array of electrodes integral with a compression knit fabric material is provided, the array of electrodes being arranged to overlie a muscle group of a subject, and each electrode of the array of electrodes being formed of a conductive knit fabric material (e.g.,). The compression knit fabric material can be part of the therapeutic garment employed for implementation of the exemplary method 800. At block 820, electric stimulation is applied to each electrode of the array of electrodes according to a prescribed treatment. In one aspect, such treatment can comprise application of waveforms having frequencies ranging from about 3 mHz to about 5 kHz. The waveforms can comprise one or more of a sine wave, a square wave, a symmetric waveform, an asymmetric waveform. In addition or in the alternative, the prescribed treatment can include synchronized or alternate (in a specific sequence) activation of one or more electrodes to produce, for example, functional movement of a body part.

Block 820 can be referred to as the applying step. An electrical therapy unit (ETU) can be a source of the electric stimulation. To permit or enhance the electric stimulation, the applying step can comprise wetting each electrode of the array of electrodes with water, an electrically conductive solution, a hypoallergenic electrically conductive solution, or a conductive gel. Utilization of such electrically conductive substances (which may be commercially available) can enhance the effectiveness of the electrical stimulation properties of the therapeutic garment. In one aspect, the applying step comprises applying a plurality of voltage waveforms effective to increase muscle strength. In another aspect, the applying step can comprise applying a plurality of voltage waveforms effective to increase muscle endurance. In yet another aspect, the applying step comprises applying a plurality of voltage waveforms effective to reduce blood clot formation in lower extremities of the subject. In still another aspect, as described herein, the applying step comprises applying a plurality of voltage waveforms effective to disuse muscle atrophy. In other aspects, the applying step can comprise applying a plurality of voltage waveforms effective to decrease rate of age related muscle loss.

One or more features (shape, size, etc.) of the therapeutic garment employed to implement the exemplary method 800 can be specific to the subject and the muscle group being treated, or submitted to the electrical stimulation, and the body part of the subject in which the muscle group is located. The body part can comprise one arm, both arms, one hand, both hands, one leg, both legs, one thigh, both thighs, a lower portion of one leg, both lower portions of both legs, one foot, two feet, neck, face, torso, hips, lower abdomen, other body parts, or any combination of the foregoing. Accordingly, in an embodiment in which the therapeutic garment is modular, after the muscle group is identified, one or more modular components (arm(s), hand(s), face and supporting straps or attachment members, torso, lower extremity(ies), lower leg(s), full leg(s) . . . ) of such garment can be identified for placement in the therapeutic garment.

Blocks 810 and 820 can be implemented for several muscles in the muscle group. Implementation can be sequential, repeating blocks 810 and 820 for two or more muscles in the muscle group. In the alternative, all or nearly all muscles in the muscle group can be stimulating at substantially the same time.

Implementation of the applying step (or block 820) can include the following. The subject's skin can be cleaned and one or more of the electrodes of the array of electrodes can be moistened with a suitable liquid (e.g., tap water) prior to applying a voltage waveform or circulating an electrical current. In one aspect, depending on the scope of treatment, for example, cleaning the subject's skin can include showering the subject, and removing oils, lotions, dirt, and other removable coatings present in the subject's skin. It should be appreciate that, in certain embodiments, presence of oil-based lotions, salves, or oils onto the subject's skin prior to treatment (or electric stimulation) may reduce effectiveness of treatment, e.g., by reducing electric conductivity among an electrode integral with the therapeutic garment and the subject's body part being treated. In a scenario in which the compression knit fabric material is part of the therapeutic garment employed to implement the exemplary method 800, the subject is dressed with such garment. Areas of the subject (such as skin or other tissue) that can be in contact with one or more electrodes can be moistened with water, an electrically conductive gel, an electrically conductive solution, or a hypoallergenic solution prior to putting on the therapeutic garment. In a scenario in which the subject is a human being, height and weight can be assessed to provide a suitable (e.g., fitting) therapeutic garment. One or more ETU lead wires can be attached to the electric therapy unit. In one aspect, the subject who wears the therapeutic garment can determine the muscle group which muscle to which the electric stimulation is applied. To at least such end, in one embodiment, the subject can identify one or more electrode sleeves for the muscle group. The one or more ETU lead wires can be attached to the one or more identified electrode sleeves having lead wires functionally connected to at least one electrode of the array of electrodes. After attachment is complete, the ETU can be turned on and current and/or voltage can be adjusted (e.g., increased) to a prescribed level suitable to achieve a desired level of contraction. For example, the ETU can be turned on and electric current (e.g., milliamps) can be increased according to the ETU manufacturer's instructions available in catalogs, owner's manuals, or other pertinent literature (e.g., therapist records). After achieving such desired level(s), the subject proceeds with treatment (e.g., a muscle training session).

As described herein, in one aspect, at the end of the therapy session, the subject can select an alternative muscle group to treat (e.g., train). In such scenario, as part of implementation of block 820, the subject can identify an electrode sleeve in the therapeutic garment that is suitable to stimulate, or treat, the alternative muscle group. After such identification, in one embodiment, the subject can shift the ETU lead wires to the electrode sleeve and can initiate a new therapy session (e.g., muscle training session).

In certain embodiments, after a predetermined time interval elapses (e.g., at the end of one hour), if the subject desires to continue therapy (e.g., muscle training), then the subject can be indicated to remoisten at least one electrode (e.g., one electrode, two electrodes, more than two electrodes, or the like) to maintain electric contact between such electrode(s) and the subject's skin that is adequate for the electric stimulation. In other embodiments, the at least one electrode can be remoistened in response to an increase is electric resistance between the at least one electrode and the subject's body part that is being electrically stimulated. It should be appreciated that such increase in the electric resistance can result in a perceived, subjective increase in discomfort experience by the subject during treatment. In the foregoing embodiments, the at least one electrode can be remoistened with water, an electrically conductive gel, an electrically conductive solution, or a hypoallergenic solution. As described herein, such electrically conductive substances can be applied by removing the therapeutic garment to gain access to the at least one electrode for the application of the water, gel, or other solutions.

Various advantages of the disclosed therapeutic garment over conventional therapeutic garments emerge from the foregoing description. As an exemplary advantage, delivery of current to tissue is simplified and comfort of a treated subject is increased. Entanglement of lead wires of the therapeutic garment of disclosure is largely mitigated or avoided. Similarly, abrupt removal of such wires from associated electrodes or from the subject's body also is reduced with respect to conventional garments, with the ensuing reduction of sudden bursts of pain to the subject.

While the systems, devices, apparatuses, protocols, processes, and methods have been described in connection with exemplary embodiments and specific illustrations, it is not intended that the scope be limited to the particular embodiments set forth, as the embodiments herein are intended in all respects to be illustrative rather than restrictive.

Unless otherwise expressly stated, it is in no way intended that any protocol, procedure, process, or method set forth herein be construed as requiring that its acts or steps be performed in a specific order. Accordingly, in the subject specification, where description of a process or method does not actually recite an order to be followed by its acts or steps or it is not otherwise specifically recited in the claims or descriptions of the subject disclosure that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification or annexed drawings, or the like.

It will be apparent to those skilled in the art that various modifications and variations can be made in the subject disclosure without departing from the scope or spirit of the subject disclosure. Other embodiments of the subject disclosure will be apparent to those skilled in the art from consideration of the specification and practice of the subject disclosure as disclosed herein. It is intended that the specification and examples be considered as non-limiting illustrations only, with a true scope and spirit of the subject disclosure being indicated by the following claims.

What is claimed is:

1. A therapeutic garment, comprising:
a compression knit fabric material;
an array of electrodes integral with the compression knit fabric material, the array of electrodes being arranged to overlie specific portions of a subject, and each electrode of the array of electrode being formed of a conductive knit fabric material, wherein the conductive knit fabric material encompasses at least a portion of a liquid absorptive material; and
a sleeve housing at least one conductive wire attached to at least one electrode of the array of electrodes.

2. The therapeutic garment of claim 1, further comprising a rivet for each electrode of the array of electrodes, wherein the rivet is configured to attach a conducting wire to an electrode associated with the rivet.

3. The therapeutic garment of claim 1, further comprising a conductive wire adhesively attached to each electrode of the array of electrodes.

4. The therapeutic garment of claim 1, wherein the conductive knit fabric material is a stretchable material.

5. The therapeutic garment of claim 1, wherein the conductive knit fabric material is a stretchable knit fabric material.

6. The therapeutic garment of claim 1, wherein the conductive knit fabric material comprises cotton fibers and metal threads.

7. The therapeutic garment of claim 1, wherein the conductive knit fabric material is a bamboo based fabric having silver fibers woven in.

8. The therapeutic garment of claim 1, wherein the therapeutic garment is modular and configured to treat a specific part of the subject.

9. The therapeutic garment of claim 1, wherein the compression knit fabric material is manufactured to exert pressure in the range from about 10 mmHg to about 65 mmHg.

10. The therapeutic garment of claim 1, wherein the compression knit fabric material is manufactured to exert pressure of at least 10 mmHg.

11. The therapeutic garment of claim 1, wherein the compression knit fabric material is manufactured to exert pressure of at least 50 mmHg.

12. The therapeutic garment of claim 1, wherein the compression knit fabric material comprises one or more of a material configured to transmit water vapor, an antibacterial material, a hypoallergenic material, or a non-abrasive material.

13. The therapeutic garment of claim 1, wherein the specific portions of the subject comprise a plurality of areas in a muscle group.

14. The therapeutic garment of claim 13, wherein at least one dimension of each electrode in the array of electrodes is determined at least in part by the muscle group.

15. A method, comprising
providing an array of electrodes integral with a compression knit fabric material, the array of electrodes being arranged to overlie a muscle group of a subject, and each electrode of the array of electrodes being formed of a conductive knit fabric material and a liquid absorptive material; and
applying electric stimulation to each electrode of the array of electrodes according to a prescribed treatment.

16. The method of claim 15, wherein the compression knit fabric material manufactured to exert pressure of at least 10 mmHg.

17. The method of claim 15, wherein the applying step comprises applying a plurality of voltage waveforms effective to increase muscle strength.

18. The method of claim 15, wherein the applying step comprises applying a plurality of voltage waveforms effective to increase muscle endurance.

19. The method of claim 15, wherein the applying step comprises applying a plurality of voltage waveforms effective to reduce blood clot formation in lower extremities of the subject.

20. The method of claim 15, wherein the applying step comprises applying a plurality of voltage waveforms effective to disuse muscle atrophy.

21. The method of claim 15, wherein the applying step comprises applying a plurality of voltage waveforms effective to decrease rate of age related muscle loss.

22. The method of claim 15, wherein the applying step comprises wetting each of the electrodes of the array of electrodes with a hypoallergenic electrically conductive solution.
